(19) United States
(12) Patent Application Publication
(10) Pub. No.: US 2008/0154360 A1
(43) Pub. Date: Jun. 26, 2008

(54) Means and Device for Compensating a Local Deformation of the Cornea of an Eye

(76) Inventors: Mark Tomalla, Mulheim (DE); Gebhard Tomalla, Essen (DE)

Correspondence Address: PROSKAUER ROSE LLP, ONE INTERNATIONAL PLACE, BOSTON, MA, 02110, US

(21) Appl. No.: 11/817,017

(22) PCT Filed: Feb. 23, 2006
(86) PCT No.: PCT/EP2006/060199

§ 371 Date: Nov. 16, 2007

(30) Foreign Application Priority Data
Feb. 25, 2005 (DE) 10 2005 009 259.4

(51) Int. Cl. A61F 2/14

(52) U.S. Cl. 623/5.11

(57) ABSTRACT

A local deformation of the cornea of an eye can be compensated by a set comprising at least two implant members for insertion into at least one receptacle inserted into the cornea, wherein at least two implant members are arranged in differing planes located one above the other. Undesirable repercussions and side effects of the intervention in the eye can be detected as early as the simulation takes place and suitable receptacles and the like can be accordingly selected in order to minimise such repercussions and side effects and, at the same time, to provide optimum compensation for the deformation of the cornea in order to compensate for the deformation.

### CROSS-REFERENCE TO RELATED APPLICATIONS

This application is a National Phase Application of International Application No. PCT/EP2006/060199, filed Feb. 23, 2006, which claims the benefit of and priority to German Application No. 10 2005 009 259.4, filed Feb. 25, 2005, which is owned by the assignee of the instant application. The disclosure of each of the above applications is incorporated herein by reference in its entirety.

### FIELD OF THE INVENTION

The present invention relates to a means and a device for compensating for a local deformation of the cornea of an eye. A means of this type and a device of this type are used for compensating for a local deformation of the cornea of the eye, especially of the human eye. However, they can also be used for treating the eye of a different creature.

### BACKGROUND OF THE INVENTION

Owing to diseases and/or injuries, the cornea of a human eye can experience reductions in thickness causing, under the influence of the aqueous humour pressure, the cornea contour, which is spherically shaped in the healthy state, to have local outwardly or inwardly shaped archings. This leads to a change in refraction with in some cases substantial distortions of the cornea (astigmatisms) for which neither glasses nor contact lenses are able to compensate satisfactorily. Severely restricted sight is the result. Such deformation of the cornea is known as keratoconus or keratectasia.

In the past, arching or bulging of the cornea has been eliminated using feed tools which are introduced from the outside and with which an approximately annular channel or tunnel is positioned purely mechanically and manually in the healthy region of the cornea. The channel or tunnel acts as a receptacle for the insertion of implants which are generally made of a transparent plastics material such as polymethyl methacrylate (PMMA). Implants of this type are known in practice as “INTACS”.

In the known method, two implants, which are arcuate in their longitudinal extension, are inserted, for example, into two corresponding receptacles, located in a common plane, in the healthy region of the cornea. The implants may in this case encompass, for example, an angular range of less than 180° and are conventionally inserted into the cornea facing one another so as at least partially to encompass the abnormally arched region of the cornea. When inserted into the cornea, the implants exert a tensile force onto the arched region of the cornea as a result of the fact that they rest against a wall of the respective receptacle. This reshapes the arching.

The known method originates from the treatment of myopia (short-sightedness) and was approved in 2004 by the US Food and Drug Administration (FDA) as a method for the treatment of keratoconus and keratectasia patients.

Operating methods of this type have the drawback, in particular, of requiring patient convalescence and aftercare lasting several months. Further drawbacks include the scarring effects resulting from lacerations and the resultant astigmatism.

There are also often introduced into the respective receptacle, during preparation, epithelial cells which can produce undesirable deposits on the INTACS. The endothelial cells extending on the inside of the cornea are also inevitably damaged during carrying-out of the known operating method owing to the tensile and compressive loads.

In the case of advanced findings, complex deimplantation/reimplantation of the cornea (perforating keratoplasty) has to be carried out.

A further drawback of this operating method is that, owing to the fixed incision width, there is no variability on introduction of the channels for the implants, and this can cause more or less pronounced stress in the treated cornea. The known method therefore often does not provide sufficiently precise compensation for the local deformation of the cornea and, in particular, the complex stress conditions occurring in the region of the deformation, so the deformation also cannot be optimally eliminated.

### SUMMARY OF THE INVENTION

The invention features, in one embodiment, a means of the type mentioned at the outset allowing improved compensation, compared to the prior art, for local deformation of the cornea of an eye. Also to be specified is a device of the type mentioned at the outset which minimises, with improved compensation for a local deformation of the cornea, undesirable side effects and/or repercussions for the patient.

In one aspect, a means for compensating for a local deformation of the cornea of an eye can include a set comprising at least two implant members for insertion into at least one receptacle inserted into the cornea, wherein at least two implant members are arranged in differing planes located one above the other.

The means according to the invention has a set for insertion into a receptacle inserted into the cornea. The set comprises in this case at least two implant members arranged in differing planes located adjacently to one another and one above the other. The set may both be of one-piece configuration and comprise two implant members which are not directly joined together. The arrangement of the implant members in two planes is maintained in this case, especially after insertion into the cornea. The implant members are provided, in particular, for insertion into the region of the cornea that is not affected by the deformation. The receptacle into which the implant members are respectively inserted may in this case, in particular, be in the form of a channel.

The fact that, in accordance with the invention, at least two implant members are arranged in differing planes allows for the complex stress conditions in a deformed cornea. Such arrangement of the implant members allows differing forces to be exerted by the implant members in differing planes of the cornea. In this way, it is possible to compensate in a more precise manner than in the prior art for the stress conditions prevailing in a deformed cornea, and thus for the deformation of the cornea. In particular, differing stress conditions prevailing in various planes of the deformation of the cornea may in this way be compensated for more effectively.

Mechanical damage to the arched and thickness-reduced region of the cornea may in this case be avoided and local arching in the cornea compensated for by implant members inserted into the cornea, especially in the environment of the arching and therefore the healthy region. Suitable matching of the formation of the receptacle in the cornea and the formation of the implant members to be inserted into the receptacle allows adjustment of the force exerted by each implant member onto the cornea and thus, in particular, onto the deformed region.

The tension properties of the implant members may be brought about during the manufacturing process by changes or deviations in the material condition, shape and volume in the extension of the element. The forces to be exerted onto the receptacle formed in the cornea may, for example, be introduced over the entire longitudinal extension of the implant members or merely in certain portions.

The tension forces exerted by the implant members may, for example, be produced from already integrated stresses or from stresses built up as a result of the insertion into the cornea. In particular, the tension forces may originate from the geometrical configuration of the implant member and the associated receptacle.

The implant members may in this case be mounted concentrically or eccentrically in the receptacles provided for them. If the implant members abut the internal or external circumference of the receptacle, the tension forces may, for example, act on a perpendicular wall of the cornea receptacle.

According to a preferred embodiment, provision is made for the implant members to be configured in such a way that, when inserted into the cornea, at least one implant member exerts a compressive force and at least one implant member exerts a tensile force onto the region of the local deformation. According to this embodiment, the implant members arranged in differing planes are able to exert onto the deformation of the cornea forces acting in differing, especially opposed, directions, i.e. tensile and compressive forces. This embodiment allows even more precise compensation for the tension conditions prevailing in the deformation.

According to a particularly preferred embodiment, the implant member exerting the compressive force may be provided for insertion into a distal plane of the cornea and the implant member exerting the tensile force may be provided for insertion into a proximal plane of the cornea. The distal plane is in this case further removed than the proximal plane from the centre of the eye. It has been found that an increased tensile stress is provided in the distal region of the deformed cornea, whereas a lower tensile stress is produced in the proximal region of the deformed cornea. This embodiment therefore provides optimum compensation for these stress conditions and thus for the deformation of the cornea.

In extreme cases, there may even be compressive stress in the proximal region of the cornea. In this case, the cornea may have in the deformed region what is known as a “neutral strand” above which there is a tensile stress and below which there is a compressive stress. In this case, it is advantageous if the implant member exerting the compressive force is provided for insertion into the cornea above the neutral strand and the implant member exerting the tensile force is provided for insertion into the cornea below the neutral strand.

In practice, it has been found to be especially suitable if at least one of the implant members is configured so as to be arcuate in the direction of its longitudinal extension. Implant members which are arcuate, especially circle-arcuate, in their longitudinal extension can be adapted particularly effectively to the geometry of the deformation of the cornea, especially to its frequently circular limitation, and apply the forces respectively associated therewith. In addition, the arcuate formation allows the implant member to be arranged so as at least partially to encompass the deformation of the cornea. A force exerted by the respective implant member onto the deformed region thus acts in a highly uniform manner on the deformed cornea region. Similarly, all of the implant members may, in particular, be of arcuate configuration.

According to a preferred teaching, the arcuate implant member may encompass an angular range of less than 360°. An implant member of this type may be introduced in a simplified manner, starting with one of its ends, into the receptacle provided in the cornea of the eye. More preferably, the arcuate implant member may encompass an angular range of less than 180°. In this way, it is possible to provide respective implant members on opposing sides of the arching of the cornea. Firstly, implant members provided on one side of the deformation may in this case be arranged in differing planes. Additionally or alternatively, however, implant members arranged on opposing sides of the deformation may each be arranged in differing planes. This large number of variations ensures that precise compensation may be provided for any type of deformation of the cornea, for example even an asymmetrical deformation.

Provision may be made for the arcuate implant member to have a second curvature, the curvature vector of which is located substantially perpendicularly to the curvature vector of the arched curvature. According to this embodiment, the implant member has not only an arcuate curvature in the longitudinal direction but also a second curvature in a direction perpendicular to the arched curvature. In this way, the implant members can be optimally adapted to the spherical contour of the cornea. The second curvature may in this case also be arcuate. In particular, the base area of the implant members may extend parallel to the surface of the cornea (both internally and externally), so deformation of the cornea is prevented transversely to the curvature of the cornea by pressing the implant members through. Obviously, the receptacle provided for each implant member may also have a corresponding second curvature. Of course, the implant member or the associated receptacle may also have further curvatures in further directions.

Since the aim is to reshape the affected thin-walled and outwardly shaped cornea region in such a way that it is optimally adapted to the remaining spherically curved surface of the cornea, use is expediently made of a plurality of control variables, such as the implant member and the receptacle, in order thus to be able purposefully to produce adequate stresses and deformation paths. In particular, it may in this case be expedient to carry out, locally and in a coordinated manner, geometric variations to the implant member and/or shape of the receptacle. Alternatively or additionally, all of the possible adaptations of the geometry of the implant members may, of course, also be carried out on the shape of the receptacle in the cornea.

According to one embodiment, at least one of the implant members may be of meandering configuration. As a result of this embodiment, it is possible, depending on the formation of the meander, to introduce forces purposefully adapted to the deformation of the cornea into the cornea through the implant member. In particular, the regions of the implant member in which the meander has turning points lead in this case to an introduction of increased force owing to reinforced supporting on the receptacle. A suitable configuration of the meander and the associated receptacle therefore allows the introduction of force into the cornea to be individually adjusted and even more flexible compensation for the deformation of the cornea thus to be achieved. The meander may in this case, in particular, have differing numbers, differing distances and/or differing positions of the alternating or turning points and/or differences in both the external and the internal radii, especially the radial differences between the largest and smallest diameter of the meander (frequency and amplitude).

Increased flexibility on introduction of compensatory forces into the cornea may also be achieved in that at least one of the implant members is of polygonal configuration. An embodiment of this type also allows the effect of the implant member to be optimally adapted to the configuration of each deformation of the cornea. A hexagon is cited merely as an example of a suitable polygonal shape.

The effect of the means may also be individually adapted in that at least one of the implant members has differing strength and/or elasticity values along its longitudinal extension. As a result of the differing strength and/or elasticity values, the tension forces exerted by the implant member onto the cornea vary as a function of the strength and elasticity of each member portion. The implant member may therefore also in this way be individually adapted to the deformation of the cornea for which compensation is to be provided.

A further embodiment provides for at least one of the implant members to have a wedge-shaped cross section. A corresponding configuration of the wedge-shaped cross section allows forces to be introduced in an especially purposeful manner. The edge of the wedge should in this case point in the respective direction in which force is intended to act, as in this direction the wedge-shaped implant member exerts a comparatively large force. The implant member may thus, for example, have a triangular cross section.

The configuration of the set with two implant members which are not directly joined together has the advantage of providing robust elements which may be optimally handled.

A one-piece configuration of the set according to the invention, on the other hand, has other advantages. Thus, in the case of a one-piece set, the compensation for the deformation of the cornea is achieved by the insertion of merely one component into the cornea. There is therefore no need to form two receptacles in the cornea and to position in a complex manner two implant members relative to each other in the cornea in order to achieve a desired introduction of force into the cornea. An alternative embodiment of the invention therefore provides for it to be possible for the set to be of one-piece configuration. In this case, the implant members of the sets are therefore integrally connected to one another.

In the case of a one-piece set, the implant members may be oriented substantially parallel to one another and be joined together by a connecting web, the connecting web being connected at one of its web ends to a leading end, in the direction of the transverse extension of the implant members, of the one implant member and at its other web end to a trailing end, in the direction of the transverse extension of the implant members, of the other implant member. This embodiment therefore allows, for example, a Z-shaped cross section of the set to be produced. The Z shape may in this case be provided prior to insertion into the cornea. However, provision may also be made for the Z shape of the set to be produced merely by tensioning on insertion into the receptacle provided in the cornea.

In any case, with a set having a Z-shaped cross section, corresponding tensioning of the set in the receptacle provided in the cornea allows both a compressive force and a tensile force to be exerted onto the deformed region of the cornea using just one component. The implant member forming the leg of the Z shape that is pointed away, when inserted, from the deformation of the cornea can in this case exert a tensile force, whereas the implant member forming the leg of the Z shape that points toward the deformation is able to exert a compressive force. In particular, provision may be made in this case for the implant member of the set that exerts the compressive force to be provided for insertion into a distal plane of the cornea, whereas the implant member exerting the tensile force is provided for insertion into a proximal plane.

According to an especially practical embodiment, at least one of the implant members may have a region which can be manipulated using magnetic forces. The implant member has in this case a region via which the implant member can be moved using magnetic forces. It is in this way possible, in an especially simple manner, to introduce the implant member into the receptacle in the cornea in that the implant member is guided using a suitable magnetic tool. The loads on the eye that are associated with the intervention are in this embodiment minimised, as insertion of the implant member into the cornea does not require any direct contact with the implant member.

A transparent material such as PMMA is preferentially used as the material for the implant members or the set. This material has proven suitable in practice. In particular, it combines good tension properties with good compatibility with the eye. However, in addition to PMMA, the material which is mainly used, use may also be made of other transparent and anatomically designed materials such as, for example, those having an irreversible shape-changing effect (memory) maintained over the entire period during which the implant member is used.

If use is made of transparent materials, for example the aforementioned PMMA, for the implant members, those having a modulus of elasticity of approx. 600 MPa and greater are preferred, as significant inherent rigidity is required to prevent the cornea itself from reshaping.

Further possible materials include those which, when implanted, have already integrated inherent stresses or the capacity to build up inherent stresses through extraneous action.

The materials used for the implant members according to the invention should in this case not change their inherent volume during their service life, i.e. in particular not swell, in order to ensure uniform action of the implant members.

Materials of this type allow the respectively required configuration, in particular geometry, to be selected in an especially individual manner without the implant members having to be manufactured specifically for treatment. However, it is also conceivable for there to be provided for the implant members a material having a reversible shape-changing effect (memory). In this case, it is possible to adapt the implant member, for example in the event of the geometry of the cornea changing a relatively long time after the treatment, to the geometry now obtaining and thus to continue using the implant member.

In another aspect, a device for compensating for a local deformation of the cornea of an eye, comprises a means for collecting property data of the local deformation, and a means for simulating the compensation for the deformation of the cornea, provided that at least two implant members are inserted into at least one receptacle inserted into the cornea, one implant member, when inserted into the cornea, exerting a tensile force and one implant member exerting a compressive force onto the region of the local deformation, comprising a means for selecting a suitable combination of a receptacle to be inserted into the cornea and a means, configured in accordance with the invention, to be inserted into the receptacle, in view of the results of the simulation, and comprising a means for inserting the receptacle into the cornea. The receptacle may in this case, in particular, be in the form of a channel.

In order to achieve, in addition to the technically possible high measurement precision in the production of the cornea receptacle and the localising thereof, optimum operative results, provisions, such as for example data collection, simulation and optimisation, have to be made prior to operation.

For this purpose, the device has a means for collecting property data, the data provided prior to compensation for the local deformation being obtained using said means. This data serves as the basis for the subsequent procedure. The means for simulating the compensation for the deformation is used to calculate the repercussions of the compensation for the cornea arching (ideal cornea arching) on the healthy environment of the cornea in order effectively and reliably to determine the position of the receptacle for the respective stabilising implant members. The simulation is carried out on the condition that at least two implant members are inserted into at least one receptacle inserted into the cornea, one implant member exerting, when inserted into the cornea, a tensile force and one implant member exerting a compressive force onto the region of the local deformation. This condition ensures precise compensation for the deformation of the cornea.

There may be provided in this case, in particular, a means which produces an optimisation or simulation model for the compensation for the deformation of the cornea. The region of the cornea to be used for this purpose is conventionally located on a diameter of approx. 8 mm based on the axis of the eye (centre point). Annular or partially annular channels or tunnels may in this case be inserted as a receptacle into the cornea and inserted into these corresponding implant members. Depending on the findings, the receptacles and implants may be mounted concentrically or eccentrically.

This region may also be taken as the starting point for the simulation of the active diameter. A quadrant circle is positioned in such a way that the cornea arching is located in a quadrant. Based on the receptacle and implant member combinations and variations, radial force action lines are defined for tension and/or compression force in each quadrant and the extent and effect thereof on the compensation for the arching are established. Equally crucial for the simulation and awareness of the above-described variables is that of the paths for reshaping of the deformation of the cornea.

The most suitable combination of the channel and implant configuration is selected and implemented based on the model of the results of directions of action and active variables in force and distance. For this purpose, the means is used for selecting a suitable combination of a receptacle to be inserted into the cornea and a means which is configured in accordance with the invention and is to be inserted into the receptacle, in view of the results of the simulation. The combination, which is the optimum combination for compensation for each deformation of the cornea, of the receptacle and means according to the invention for compensating for a local deformation of the cornea is selected based on the results of the simulation. Finally, there is provided a means for inserting into the cornea the receptacle which is suitable for the respectively selected means. The means is then inserted into this receptacle.

The device according to the invention allows undesirable repercussions and side effects of the intervention into the eye to be detected as early as the simulation takes place and accordingly suitable receptacles and means to be selected in order to minimise such repercussions and side effects. At the same time, optimum compensation for the deformation of the cornea is achieved by the means according to the invention for compensating for the deformation.

The data calculated by the means for collecting the property data may, in particular, be data concerning the thickness, elasticity, ductility and/or strength of the cornea in the deformed region (endothelial microscopy) and/or data concerning the geometry and/or the position of the local deformation, especially in relation to the axis of the eye and/or the healthy cornea region. All of this data is relevant to the selection of suitable receptacles and implant members. Following the topography and determination of the geometric and material-specific data of the cornea, an optimisation or simulation model is then produced for compensating for the deformation of the cornea.

The non-deformed region of the cornea may also be examined, firstly to avoid undesirable side effects on the healthy region during treatment, but also to calculate the required forces which are to be introduced and are tolerable for the cornea. The device may have for this purpose a means for collecting the property data of the non-deformed region of the cornea. All of the property data collected for the deformed region may in this case also be collected for the healthy region of the cornea. The spherical formation of the cornea in the healthy region may, for example, also be detected in this case.

The device may also have, prior to the inserting of the receptacle, a means for marking the region of the cornea that is provided for inserting of the receptacle. Once a suitable receptacle has been selected and positioned in the cornea, this means is therefore used for marking the region of the cornea that is to be provided with the receptacle before the receptacle is inserted. A marking process of this type is interconnected in the event of non-negligible deviations in the contour of the cornea during docking the means for inserting the receptacle to the eye to be operated on. In particular, if the means for inserting the receptacle is docked to the eye using an adapter, deviations in the contour of the cornea may result from the pressure ratios between the low-pressure to be built up in the adapter and the contact pressure required for positioning the adapter onto the eye.

The marking means can, for example, transmit the position and shape data resulting from the simulation phase to the normal actual state of the eye in such a way that the coordinates, which ideally correspond to the selected implant member, of the receptacle course are applied to the cornea, for example, using a template which is preferably made of PMMA and is adapted to the cornea once the deformation of the cornea has been compensated for. A corresponding solution may be achieved using a biometrically exact detection system integrated in the field of excimer lasers.

If the means for inserting the receptacle is then docked to the eye, it may be checked to what extent the ideal course of the receptacle has been achieved or the means for inserting the receptacle has to be corrected in order to ensure this.

It is important that this condition is adhered to, as failure to do so will directly impair the success of the operation as a result of the fact that the selected implant members cannot function as intended. Thus, for example, inserting of the receptacle causes, in the case of an excessively curved cornea, a desired diameter of a receptacle, for example, to be too large and, in the case of a cornea which is pressed too flat, a desired diameter of a receptacle to be too small. Imprecise alignment leads not only to functional problems but also to implant problems and undesirable consequences thereof.

According to a preferred embodiment, the means for inserting the receptacle may have an adapter to be attached to the eye for fixing the eye during the inserting of the receptacle. For implanting the set, it may be expedient if an adapter is used to support the desired shape of the region of the cornea in which implantation is not carried out. An adapter of this type may, for example, be configured in a similar manner to a laser adapter known per se that is adapted to the patient interface (PI).

The adapter may be in the form of a truncated cone-shaped funnel. The adapter proposed in this case may be of similar configuration to adapters known per se for these purposes. However, it has on its circumference a sufficiently large opening to allow the implant member to be inserted into the receptacle without obstruction via the section of the receptacle when the adapter is attached. In order to provide the operator with optimum visibility of the operating field, a wholly transparent material such as, for example, PMMA is, in particular, suitable as the material for this adapter.

The engagement opening in the adapter for inserting the implant member should in this case, on the one hand, be sufficiently large to ensure trouble-free implantation. On the other hand, a contact surface between the adapter and cornea that is as large as possible is desirable in order to be able sufficiently to stabilise the shape of the cornea. In practice, a suitable compromise has to be found between these two aims.

The means for inserting the receptacle in the cornea may have a laser, especially a femtosecond laser. Owing to their good focusability, lasers allow the receptacle to be inserted with particular precision in the cornea. The good focusability also means that the repercussions brought about by the coupling of energy into the tissue adjacent to the focus of the laser during inserting of the receptacle are minimal. Undesirable repercussions on the cornea tissue may thus be substantially avoided. Femtosecond lasers are pulsed lasers, the pulse durations of which are in the range of femtoseconds. Owing to the short pulse duration of lasers of this type pulsed at such high frequencies, energy from the laser is coupled merely very briefly into the processed tissue. There is therefore no substantial spread of coupled-in energy into adjacent tissue. Undesirable repercussions, caused by heating, on the cornea tissue adjacent to the tissue provided for inserting the receptacle are thus further minimised.

### BRIEF DESCRIPTION OF THE DRAWINGS

The invention will be described in greater detail hereinafter with reference to exemplary embodiments illustrated in the drawings, in which:

FIG. 1 is a partial cross section of the cornea of a human eye with a means according to the invention inserted into the cornea in accordance with a first exemplary embodiment,

FIG. 2 is a plan view of a detail of an implant member according to the invention in accordance with a further exemplary embodiment,

FIG. 3 is a partial cross section of the cornea of a human eye with a means according to the invention inserted into the cornea in accordance with a further exemplary embodiment,

FIG. 4 is a partial cross section of the cornea of a human eye with a deformation of the cornea for which compensation has been provided in accordance with a first exemplary embodiment, and

FIG. 5 is a partial cross section of the cornea of a human eye with a deformation of the cornea for which compensation has been provided in accordance with a further exemplary embodiment.

### DESCRIPTION OF THE INVENTION

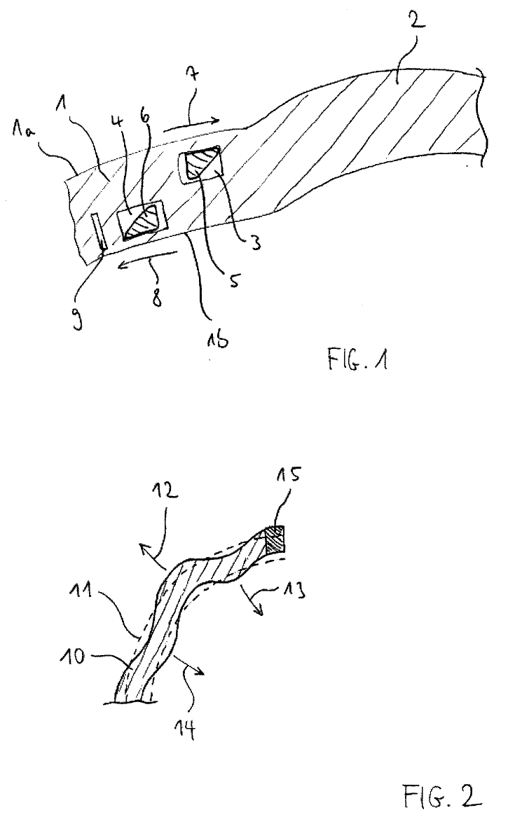

FIG. 1 is a cross section of a detail of the cornea 1 of a human eye. The cornea has a distal surface 1a and a proximal surface 1b facing the retina of the eye. In the cornea 1, a local deformation 2 is formed in the form of a circularly delimited arching. In the non-deformed, healthy region of the cornea 1, two channel-like receptacles 3, 4 have been formed using a femtosecond laser.

The receptacles 3, 4 have a rectangular cross section and are of circle-arcuate shape in their longitudinal extension. They each encompass an angular range of less than 180°. The centre of curvature of the receptacles 3, 4 is located in this case approximately on an axis (not shown) running through the centre of the deformation 2. The receptacle 3 is inserted in a distal plane of the cornea 1 and the receptacle 4 is inserted in a proximal plane of the cornea 1.

For compensating for the deformation 2 of the cornea 1, implant members 5, 6, each made of PMMA, are inserted into the receptacles 3, 4. The implant members 5, 6 form a set inserted into the cornea 1, the implant members 5, 6 being arranged in differing planes defined by the receptacles 3, 4. The implant members 5, 6, like the receptacles 3, 4, have a shape which is arcuate in their longitudinal extension and enclose an angular range of less than 180°. The implant members 5, 6 each have a wedge-shaped cross section. The edge of the wedge of the implant member 5 inserted into the distal plane of the cornea 1 is in this case oriented toward the deformation 2, whereas the edge of the wedge of the implant member 6 inserted into the proximal plane of the cornea 1 faces away from the deformation 2. The implant member 5 inserted into the distal plane has a smaller curvature than the associated receptacle 3, whereas the implant member 6 inserted into the proximal plane has a larger curvature than the associated receptacle 4.

On insertion of the implant members 5, 6 into the associated receptacles 3, 4, the implant members 5, 6 are accordingly tensioned in the cornea 1. Owing to the respective ratio of curvature of the implant member 5, 6 to the receptacle 3, 4, the tensioning causes the implant member 5 inserted into the distal plane to exert substantially a compressive force onto the region of the local deformation 2 of the cornea 1, whereas the implant member 6 inserted into the proximal plane produces substantially a tensile force onto the region of the local deformation 2 of the cornea 1. The compressive or tensile forces exerted by the implant members 5, 6 are indicated in FIG. 1 by arrows 7, 8. On the opposing side (not shown) of the deformation 2, two corresponding implant members are inserted into corresponding receptacles in the cornea 1.

Owing to the forces introduced by the implant members into the cornea 1 and, in particular, into the region of the deformation 2, the local deformation 2 is reshaped in the desired manner.

The implant members 5, 6 may have a second curvature, the curvature vector of which is located substantially perpendicularly to the curvature vector of the arched curvature oriented in the longitudinal direction oriented into the plane of the drawing. In this way, it is possible to adapt the implant members 5, 6 to the generally spherical formation of the cornea 1, thus avoiding undesirable stresses on insertion of the implant members 5, 6. In addition, the degree to which the cornea is reshaped can be enlarged in that, for example, the implant member 6 for the proximal region has concavely arched roundings (which may not be seen in the present case) on the base area and the channel 4 is triangular in cross section. The roundings may in this case be distributed uniformly over the length of the implant member 6 or restricted to specific portions in order locally to achieve a particular compensatory effect.

In order to allow the cornea 1 to compensate for any compression caused by the inserted implant members 5, 6, a relief chamber 9 is inserted in the cornea 1. The relief chamber 9 extends on the side of the receptacle 4 that is remote from the deformation 2 substantially parallel to the receptacle 4.

FIG. 2 is a plan view of a detail of an implant member 10 according to a further exemplary embodiment. The implant member 10 extends in an arcuate manner over an angular range of less than 180° and is of meandering configuration.

At one of its ends, the implant member 10 has a magnet-sensitive region 15 which can be manipulated using magnetic forces. It is therefore possible to guide the implant member 10 into a receptacle provided for the implant member 10 using, for example, magnetic forces exerted by a permanent magnet onto the region 15 without direct contact with the implant member 10 being required for insertion into the receptacle. Impairments of the eye during insertion of the implant member 10 are in this way largely avoided.

If the implant member 10 is inserted into an arcuate receptacle which is of non-meandering configuration, like the receptacle 11 indicated by broken lines in FIG. 2, the implant member 10 introduces into the cornea a differing force, depending on the shape of the meander. There is provided in this case a second implant member (not shown in FIG. 2) which is inserted into a second receptacle arranged in a plane differing from the plane of the receptacle 11. The second implant member and the second receptacle may in this case correspond in their configuration substantially to the first implant member 10 and the first receptacle 11. However, the implant members and/or the receptacles may also be of differing configuration. Obviously, still further implant members may be provided for insertion into the cornea.

In those regions in which the meandering implant member 10 has a greater curvature than the receptacle 11, the implant member 10 exerts, when inserted into the cornea, a tensile force, pointed away from the centre of the local deformation (not shown) of the cornea, onto the deformed region of the cornea. This is indicated in FIG. 2 by the arrow 12. Conversely, in those regions in which the meandering implant member 10 has a smaller or even opposed curvature in relation to the receptacle 11, the implant member 10 exerts, when inserted into the cornea, a compressive force oriented toward the centre of the local deformation (not shown) of the cornea.

This is indicated in FIG. 2 by the arrows 13, 14. The second implant member (not shown in FIG. 2) exerts, when inserted into the cornea, corresponding forces onto the cornea. The cooperation of the forces introduced into the cornea in differing planes by the implant members provides particularly precise reshaping of the local deformation of the cornea. The result of the reshaping may be particularly purposefully influenced by corresponding formation of the meander. This embodiment is an example of a basically punctiform or local introduction of forces for both tensile and compressive forces.

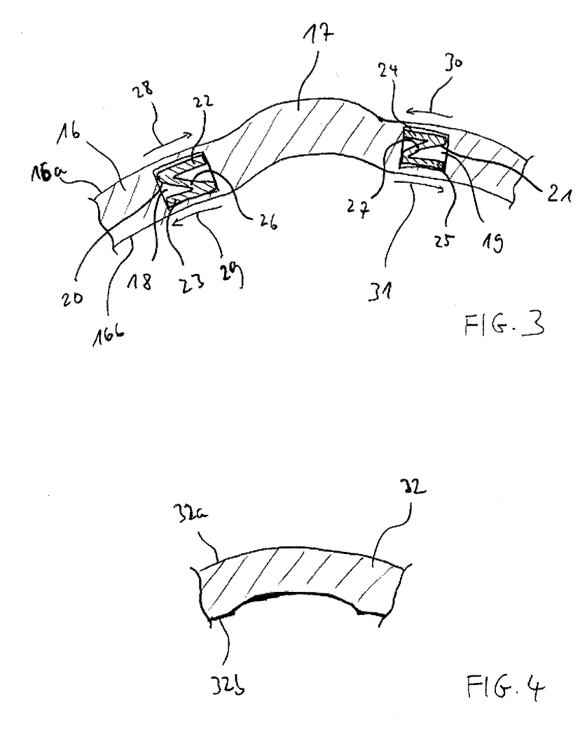

FIG. 3 is a cross section of a detail of the cornea 16 of a human eye. Again, the cornea 16 has a distal surface 16a and a proximal surface 16b facing the retina of the eye.

Formed on the cornea 16 is a local deformation 17 in the form of a circularly delimited arching. Two receptacles 18, 19 are formed in the healthy region of the cornea 16 on either side of the deformation 17.

The channel-like receptacles 18, 19 have a rectangular cross section and are arcuate, especially circle-arcuate, in their longitudinal extension. They each enclose an angular range of less than 180°. The centre of curvature of the receptacles 18, 19 is in this case located approximately on an axis (not shown) running through the centre of the deformation 17.

For compensating for the deformation 17, two sets 20, 21, each of one-piece configuration, are inserted into the receptacles 18 and 19. The sets 20, 21 each comprise two implant members 22, 23, 24, 25. The implant members of a set 20, 21 are oriented substantially parallel to one another and joined together by a respective connecting web 26, 27. The connecting web 26, 27 is connected at one of its web ends to a leading end, in the direction of the transverse extension of the implant members 22, 23, 24, 25, of the one implant member 22, 24 and at its other web end to a trailing end, in the direction of the transverse extension of the implant members 22, 23, 24, 25, of the other implant member 23, 25. When inserted into the cornea 16, the sets 20, 21 of this embodiment each have a Z-shaped cross section.

The leg, formed by the implant member 22, 24 respectively inserted into a distal plane, of the Z shape is in this case oriented toward the deformation 17. The leg, formed by the implant member 23, 25 respectively inserted into a proximal plane, of the Z shape points, on the other hand, away from the deformation 17.

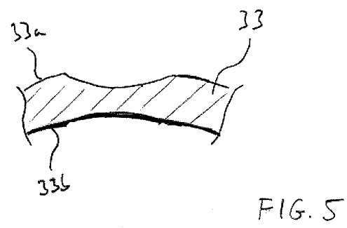

The sets 20, 21 are inserted into the cornea 16 under tension in such a way that the distal implant members 22, 24 exert a compressive force onto the deformation 17, whereas the proximal implant members 23, 25 exert a tensile force onto the deformation. This is indicated in FIG. 3 respectively by the arrows 28, 29, 30, 31. It is thus possible, using a one-piece set 20, 21, to exert both a tensile force and a compressive force onto the local deformation 17 and to compensate for the deformation especially effectively.

Whereas in FIGS. 1 and 3 the local deformation of the cornea is respectively shown when not yet compensated for, FIGS. 4 and 5 each show a detail of the cross section of the cornea 32, 33 of a human eye, there being shown in each case the state of the cornea 32, 33 in which compensation has already been provided for a previously existing deformation of the cornea. The cornea 32, 33 has a respective distal surface 32a, 33a and a proximal surface 32b, 33b facing the retina of the eye.

Depending on the configuration of the sets for insertion into the cornea 32, 33 and the receptacles provided for this purpose in the cornea, the deformation may, for example, be corrected in such a way that, in the compensated state, the distal surface of the deformation is adapted to the distal surface 32a of the healthy cornea 32. This is illustrated in FIG. 4. The state is achieved, in particular, if implant members provided in a distal plane of the cornea 32 exert merely a low compressive force onto the deformation, whereas implant members provided in a proximal plane of the cornea 32 exert a dominant tensile force onto the deformation. In this case, there may remain in the region of the proximal surface 32b of the cornea, even after the compensation, a minor deformation, although this does not cause any substantial drawbacks in relation to sight.

Alternatively, it is also possible to adapt the proximal surface of the deformation to the proximal surface 33b of the healthy cornea 33. This is the stablest state of the compensated-for deformation. This state is shown in FIG. 5. Depending on the intensity of the deformation, there may be produced in the region of the previous deformation a slight arching of the distal surface 33a of the cornea 33 that can be compensated for using an appropriate lens. The compensation illustrated in FIG. 5 is achieved, in particular, in that a correspondingly configured implant member exerts a stronger compressive force in a distal plane of the cornea 33.

### LIST OF REFERENCE NUMERALS

1 Cornea
1a Distal surface of the cornea
1b Proximal surface of the cornea
2 Local deformation of the cornea
3 Receptacle
4 Receptacle
5 Implant member
6 Implant member
7 Arrow
8 Arrow
9 Relief chamber
10 Implant member
11 Receptacle
12 Arrow
13 Arrow
14 Arrow
15 Region which can be magnetically manipulated
16 Cornea
16a Distal surface of the cornea
16b Proximal surface of the cornea
17 Local deformation of the cornea
18 Receptacle
19 Receptacle
20 Set
21 Set
22 Implant member
23 Implant member
24 Implant member
25 Implant member
26 Connecting web
27 Connecting web
28 Arrow
29 Arrow
30 Arrow
31 Arrow
32 Cornea
32a Distal surface of the cornea
32b Proximal surface of the cornea
33 Cornea
33a Distal surface of the cornea
33b Proximal surface of the cornea

1. A means for compensating for a local deformation of the cornea of an eye, having a set comprising at least two implant members for insertion into at least one receptacle formed in the cornea, wherein at least two implant members are arranged in differing planes located one above the other.

2. The means according to claim 1, characterised in that the implant members are configured in such a way that, when inserted into the cornea, at least one implant member exerts a compressive force and at least one implant member exerts a tensile force onto the region of the local deformation.

3. The means according to claim 2, characterised in that the implant member exerting the compressive force is provided for insertion into a distal plane of the cornea and the implant member exerting the tensile force is provided for insertion into a proximal plane of the cornea.

4. The means according to claim 1, characterised in that at least one of the implant members is configured so as to be arcuate in the direction of its longitudinal extension.

5. The means according to claim 4, characterised in that the implant member encompasses an angular range of less than 360°.

6. The means according to claim 5, characterised in that the implant member encompasses an angular range of less than 180°.

7. The means according to claim 4, characterised in that the arcuate implant member has a second curvature, the curvature vector of which is located substantially perpendicularly to the curvature vector of the arched curvature.

8. The means according to claim 1, characterised in that at least one of the implant members is of meandering configuration.

9. The means according to claim 1, characterised in that at least one of the implant members is of polygonal configuration.

10. The means according to claim 1, characterised in that at least one of the implant members has differing strength and/or elasticity values along its longitudinal extension.

11. The means according to claim 1, characterised in that at least one of the implant members has a wedge-shaped cross section.

12. The means according to claim 1, characterised in that the set (20, 21) is of one-piece configuration.

13. The means according to claim 12, characterised in that the implant members are oriented substantially parallel to one another and are joined together by a connecting web, the connecting web being connected at one of its web ends to a leading end, in the direction of the transverse extension of the implant members, of the one implant member and at its other web end to a trailing end, in the direction of the transverse extension of the implant members, of the other implant member.

14. The means according to claim 1, characterised in that at least one of the implant members has a region which can be manipulated using magnetic forces.

15. The device for compensating for a local deformation of the cornea of an eye, comprising:
a means for collecting property data of the local deformation;
a means for simulating the compensation for the deformation of the cornea, provided that at least two implant members are inserted into at least one receptacle formed in the cornea, one implant member, when inserted into the cornea, exerting a tensile force and one implant member exerting a compressive force onto the region of the local deformation;
a means for selecting a suitable combination of a receptacle to be formed in the cornea and
a means for compensating for the deformation of the cornea, having a set comprising the at least two implant members for insertion into the at least one receptacle formed in the cornea, in view of the results of the simulation; and
a means for inserting the receptacle into the cornea.

16. The device according to claim 15, characterised in that the property data is data concerning at least one of (i) at least one of thickness, elasticity, ductility and strength of the cornea in the deformed region and (ii) at least one of data concerning the geometry and the position of the local deformation.

17. The device according to claim 15, characterised in that there is provided a means for collecting the property data of the non-deformed region of the cornea.

18. The device according to claim 15, characterised in that prior to the insertion of the receptacle there is provided a means for marking the region of the cornea that is provided for the insertion of the receptacle.

19. The device according to claim 15, characterised in that the means for inserting the receptacle has an adapter to be attached to the eye for fixing the eye during the insertion of the receptacle.

20. The device according to claim 15, characterised in that the means for inserting the receptacle in the cornea comprises a laser, especially a femtosecond laser.
